(19) United States
(12) Patent Application Publication
(10) Pub. No.: US 2019/0024123 A1
(43) Pub. Date: Jan. 24, 2019

(54) Genome Editing In Bacillus Host Cells

(71) Applicant: Novozymes A/S, Bagsvaerd (DK)

(72) Inventors: Randy Berka, Davis, CA (US); Barbara Cherry, Winters, CA (US)

(73) Assignee: Novozymes A/S, Bagsvaerd (DK)

(21) Appl. No.: 16/065,950

(22) PCT Filed: Jan. 6, 2017
(86) PCT No.: PCT/EP2017/050252

§ 371 Date: Jun. 25, 2018

Related U.S. Application Data
(60) Provisional application No. 62/276,402, filed on Jan. 8, 2016.

(51) Int. Cl. C12N 15/90; C12N 9/22; C12N 15/11; C12N 15/75

(52) U.S. Cl. CPC C12N 15/902; C12N 9/22; C12N 2310/20; C12N 15/75; C12N 2800/80; C12N 15/11

(57) ABSTRACT

The present invention relates to methods for modifying the genome of a Bacillus host cell by employing a Class II Cas9 enzyme with only one active nuclease domain, e.g. the S. pyogenes Cas9 nickase, together with a suitable guide RNA for each target sequence to generate a site-specific nick in at least one genome target sequence followed by the repair of the nick(s) via integration of one or more modified modified donor part of the Bacillus host cell genome through classical double homologous recombination on each side of the nick(s).

### REFERENCE TO A SEQUENCE LISTING

This application contains a Sequence Listing in computer readable form, which is incorporated herein by reference.

### FIELD OF THE INVENTION

We have observed that expression of an intact Streptococcus pyogenes Cas9 enzyme was lethal in a Bacillus subtilis host cell. However, by expressing a single-strand cutting variant termed Cas9 nickase (Cas9n) of the S. pyogenes Cas9 enzyme, we successfully edited the B. subtilis genome with efficiencies approaching 50%. Based on these results, we propose that Class II Cas9 nickase systems may be deployed as genome editing tools in other Bacillus species.

The present invention relates to methods for modifying the genome of a Bacillus host cell by employing a Class II Cas9 enzyme with only one active nuclease domain, e.g. the S. pyogenes Cas9 nickase, together with a suitable guide RNA for each target sequence to generate a site-specific nick in at least one genome target sequence followed by the repair of the nick(s) via integration of one or more modified modified donor part of the Bacillus host cell genome through classical double homologous recombination on each side of the nick(s).

### BACKGROUND OF THE INVENTION

The so-called CRISPR (clustered regularly interspaced short palindromic repeats) Cas9 genome editing system originally isolated from S. pyogenes has been widely used as a tool to modify the genomes of a number of eukaryotes. However, only a few publications have reported the use of this editing system in bacteria.

The Cas9 enzyme has two RNA-guided DNA endonuclease domains capable of targeting specific genomic sequences. The system has been described extensively for editing genomes in a variety of eukaryotes (Doudna, J. A. and E. Charpentier, Genome editing. The new frontier of genome engineering with CRISPR-Cas9. Science, 2014. 346(6213): p. 1258096), E. coli (Jiang, W., et al., RNA-guided editing of bacterial genomes using CRISPR-Cas systems. Nat Biotechnol, 2013. 31(3): p. 233-9), yeast (DiCarlo, J. E., et al., Genome engineering in Saccharomyces cerevisiae using CRISPR-Cas systems. Nucleic Acids Res, 2013. 41(7): p. 4336-43), Lactobacillus (Oh, J. H. and J. P. van Pijkeren, CRISPR-Cas9-assisted recombineering in Lactobacillus reuteri. Nucleic Acids Res, 2014. 42(17): p. e131) and filamentous fungi such as Trichoderma reesei (Liu, R., et al., Efficient genome editing in filamentous fungus Trichoderma reesei using the CRISPR/Cas9 system. Cell Discovery, 2015. 1) and Aspergillus niger (Nødvig, C. S., et al., A CRISPR-Cas9 System for Genetic Engineering of Filamentous Fungi. PLoS ONE, 2015. 10(7): p. e0133085).

The power of the Cas9 system lies in its simplicity to target and edit up to a single base pair in a specific gene of interest. In addition, it is possible to target multiple genes for modification (multiplexing) in a single reaction, generate insertions and deletions, as well as silence or activate genes. In 2012, The CRISPR-Cas9 protein was shown to be a dual-RNA guided endonuclease protein (Jinek, M., et al., A programmable dual-RNA-guided DNA endonuclease in adaptive bacterial immunity. Science, 2012. 337(6096): p. 816-21.). Further development for utilization the CRISPR-Cas9 as a genome editing tool has led to the engineering of a single guided RNA molecule that guides the endonuclease to its DNA target. The single guide RNA retains the critical features necessary for both interaction with the Cas9 protein and further targeting to the desired nucleotide sequence. When complexed with the RNA molecule, the Cas9 protein will bind DNA sequence and create a double stranded break using two catalytic domains. When engineered to contain a single amino acid mutation in either catalytic domain, the Cas9 protein functions as a nickase, a variant protein with single stranded cleavage activity. Genome editing in Clostridium cellulyticum via CRISPR-Cas9 nickase was recently demonstrated by Xu et al. (Xu, T., et al., Efficient Genome Editing in Clostridium cellulolyticum via CRISPR-Cas9 Nickase. Appl Environ Microbiol, 2015. 81(13): p. 4423-31.).

A multitude of patent publications relate to the CRISPR-Cas9 genome editing system, but until now its successful application in Bacillus host cells has not been reported.

### SUMMARY OF THE INVENTION

We have observed that expression of an intact Streptococcus pyogenes Cas9 enzyme was lethal in a Bacillus subtilis host cell. Herein we show that by expressing a single-strand cutting variant termed Cas9 nickase (Cas9n) of the S. pyogenes Cas9 enzyme, we could successfully edit the B. subtilis genome with efficiencies approaching 50%. Based on the results herein results, we propose that Class II Cas9 nickase systems may be deployed as genome editing tools in Bacillus host cells.

Accordingly, in a first aspect the invention relates to methods for modifying the genome of a Bacillus host cell, said method comprising the steps of:

A. providing a Bacillus host cell comprising:
a) at least one genome target sequence to be modified, wherein each target sequence is flanked by a functional PAM sequence for a Class-II Cas9 enzyme;
b) a variant of the Class-II Cas9 enzyme having only one active nuclease domain,
c) a single-guide RNA or a guide RNA complex for each target sequence to be modified, said RNA or RNA complex comprising:
   i) a first RNA comprising 20 or more nucleotides that are at least 80% complementary to and capable of hybridizing to the at least one genome target sequence to be modified and comprising a tracr mate sequence, and
   ii) a second RNA comprising a tracr sequence complementary to and capable of hybridizing with the tracr mate sequence; and
d) at least one polynucleotide construct comprising one or more modified donor part of the Bacillus host cell genome, said donor part comprising the at least one genome target sequence having the desired nucleotide modification(s) as well as at least 70 unmodified nucleotides flanking the modification(s) on each side;
   wherein the 20 or more nucleotides of the first RNA hybridize with the at least one genome target sequence and wherein the variant Class-II Cas9 enzyme interacts with the single-guide RNA or the guide RNA complex and nicks the at least one genome target sequence,
   whereafter the one or more modified donor part of the Bacillus host cell genome is inserted into the genome by a homologous recombination event on each side of the nick, thereby introducing the desired modification(s) into the genome; and
   selecting a Bacillus host cell, wherein the at least one genome target sequence has been modified.

### BRIEF DESCRIPTION OF THE FIGURES

FIG. 1 shows two representations of the temperature-sensitive plasmid pBM367b which contains the trpC guideRNA under transcriptional control of the strong PscBAN-rbs promoter and the erythromycin resistance gene (ery) for selection in Bacillus.

FIG. 2 shows two representations of the temperature-sensitive plasmid pBM373 which contains the trpC gRNA under transcriptional control of the strong PscBAN-rbs promoter, the erythromycin resistance gene, and a ˜600 bp donor DNA fragment which when incorporated at the trpC locus would render the cells Trp+.

FIG. 3A shows a schematic representation for the Cas9 expression construct in strain BaC0291.

FIG. 3B shows a BaC0291 cell lysate visualized by SDS-PAGE, where the the intense band at 158 kDA suggests expression of the Cas9 protein verified using alpha-Cas9-specific antibodies in a Western blot.

FIG. 4 shows the in-vitro digestion of the trpC target DNA by in-vitro transcribed trpC CRISPR guide RNA complexed with purified Cas9 protein. Here, the 2 kb target is cleaved into two DNA fragments as expected. The in vitro reaction does not show complete cleavage of the target DNA, likely due to the non-optimal ratio of Cas9:gRNA:DNA present in the reaction. However, the cleavage inefficiency may also be due to a non-optimal guide RNA target sequence.

FIG. 5 shows a photo of an agarose electrophoresis gel, from which the trpC CRISPR guide RNA transcription was verified.

FIG. 6 shows a simplified schematic demonstrating the method of the present invention for Cas9n genome editing in B. subtilis.

FIG. 7 shows a snapshot from sequencing analysis at the trpC locus. B. subtilis 168.DELTA.4 unedited genome sequence is aligned with sequencing fragments from one isolate at the trpC locus. The underlined sequences indicate the targeted 3 base pair insertion and silent mutation of the PAM sequence.

### DEFINITIONS

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity” or “sequence complementarity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity (or corresponding sequence complementarity) between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

To determine the % complementarity of two complementary sequences, one of the two sequences needs to be converted to its complementary sequence before the % complementarity can then be calculated as the % identity between the the first sequence and the second converted sequences using the above-mentioned algorithm.

Variant: The term “variant” means a polypeptide comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position, e.g., 1-5 amino acids, adjacent to the amino acid occupying a position.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

### DETAILED DESCRIPTION OF THE INVENTION

In a first aspect the invention relates to methods for modifying the genome of a Bacillus host cell, said method comprising the steps of:

B. providing a Bacillus host cell comprising:
a) at least one genome target sequence to be modified, wherein each target sequence is flanked by a functional PAM sequence for a Class-II Cas9 enzyme;
b) a variant of the Class-II Cas9 enzyme having only one active nuclease domain,
c) a single-guide RNA or a guide RNA complex for each target sequence to be modified, said RNA or RNA complex comprising:
   i) a first RNA comprising 20 or more nucleotides that are at least 80% complementary to and capable of hybridizing to the at least one genome target sequence to be modified and comprising a tracr mate sequence, and
   ii) a second RNA comprising a tracr sequence complementary to and capable of hybridizing with the tracr mate sequence; and
d) at least one polynucleotide construct comprising one or more modified donor part of the Bacillus host cell genome, said donor part comprising the at least one genome target sequence having the desired nucleotide modification(s) as well as at least 70 unmodified nucleotides flanking the modification(s) on each side;
   wherein the 20 or more nucleotides of the first RNA hybridize with the at least one genome target sequence and wherein the variant Class-II Cas9 enzyme interacts with the single-guide RNA or the guide RNA complex and nicks the at least one genome target sequence,
   whereafter the one or more modified donor part of the Bacillus host cell genome is inserted into the genome by a homologous recombination event on each side of the nick, thereby introducing the desired mofication(s) into the genome; and
   selecting a Bacillus host cell, wherein the at least one genome target sequence has been modified.

### Bacillus Host Cells

The present invention also relates to recombinant Bacillus host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication.

In a preferred embodiment of the present invention, the Bacillus host cell is chosen from the group of Bacillus species consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

### Genome Target Sequence

The at least one genome target sequence to be modified by the methods of the invention is at least 20 nucleotides in length in order to allow its hybridization to the corresponding 20 nucleotide sequence of the guide RNA. The at least one genome target sequence to be modified can be located anywhere in the genome but will often be within a coding sequence or open reading frame.

The at least one genome target sequence to be modified need to have a suitable protospacer adjacent motif (PAM) located next to it to allow the corresponding Class-II Cas9 nickase enzyme to bind a nick the target. The PAM for the S.pyogenes Cas9 enzyme has been reported to be a ccc triplet on the guide RNA complementary strand (the hybridizing strand of the target sequence). See Jinek M. et al., A programmable dual-RNA-guided DNA endonuclease in adaptive bacterial immunity. Science. 2012; 337:816-21.

For an overview of other PAM sequences, see, for example, Shah, S. A. et al, Protospacer recognition motifs, RNA Biol. 2013 May 1; 10(5): 891-899.

Accordingly, in a preferred embodiment of the invention, the at least one genome target sequence to be modified comprises at least 20 nucleotides; preferably the at least one genome target sequence to be modified is comprised in an open reading frame encoding a polypeptide.

### Class-II Cas9 Nickase

Several Class-II Cas9 analogues or homologues are known and more are being discovered almost monthly as the scientific interest has surged over the last few years; a review is provided in Makarova K. S. et al, An updated evolutionary classification of CRISPR-Cas systems, 2015, Nature vol. 13: 722-736.

The Cas9 enzyme of Streptomyces pyogenes is a model Class-II Cas9 enzyme and it is to-date the best characterized. A variant of this enzyme was developed which has only one active nuclease domain (as opposed to the two active domains in the wildtype enzyme) by substituting a single amino acid, aspartic acid for alanine, in position 10: D10A. It is expected that other Class-II Cas9 enzymes may be modified similarly.

Accordingly, in a preferred embodiment, the variant of the Class-II Cas9 enzyme having only one active nuclease domain comprises a substitution of aspartic acid for alanine in the amino acid position corresponding to position 10, D10A, in the Streptomyces pyogenes Cas9 amino acid sequence shown in SEQ ID NO:8.

In an even more preferred embodiment, the variant of the Class-II Cas9 enzyme having only one active nuclease domain has the amino acid sequence shown in SEQ ID NO:22.

### Guide RNA

The guide RNA in CRISPR-Cas9 genome editing constitutes the re-programmable part that makes the system so versatile. In the natural S. pyogenes system the guide RNA is actually a complex of two RNA polynucleotides, a first crRNA containing about 20 nucleotides that determine the specificity of the Cas9 enzyme as well as the tracr RNA which hybridizes to the cr RNA to form an RNA complex that interacts with Cas9. See Jinek M. et al., A programmable dual-RNA-guided DNA endonuclease in adaptive bacterial immunity. Science. 2012; 337:816-21. The terms crRNA and tracrRNA are used interchangeably with the terms tracr-mate RNA and tracr RNA herein.

Since the discovery of the CRISPR-Cas9 system single polynucleotide guide RNAs have been developed and successfully applied just as effectively as the natural two part guide RNA complex.

In a preferred embodiment, the single-guide RNA or RNA complex comprises a first RNA comprising 20 or more nucleotides that are at least 85% complementary to and capable of hybridizing to the at least one genome target sequence; preferably the 20 or more nucleotides are at least 90%, 95%, 97%, 98%, 99% or even 100% complementary to and capable of hybridizing to the at least one genome target sequence.

In another preferred embodiment, the Bacillus host cell comprises a single-guide RNA comprising the first and second RNAs in the form of a single polynucleotide and wherein the tracr mate sequence and the tracr sequence form a stem-loop structure when hybridized with each other.

### Modified Donor Part of the Genome

The methods of the intant invention rely on the integration of a modified piece of Bacillus genomic DNA back into the genome to replace a DNA section in the genome that contains the nicked target sequence. This integration happens via a classical Campbell-type homologous recombination event on each side of the nicked target sequence, or actually just on each side of the nick. This double homologous recombination requires sufficient wildtype donor genomic DNA flanking the modified sequence to enable effective recombination. Bacillus has been reported to require approximately 70 nucleotides of identical sequences to allow homologous recombination between the genome and a plasmid (Khasanov et al. Mol Gen Genet (1992) 234:494-497). So the modified donor DNA for integration should contain the actual modification plus around 70 nucleotides on each side for successful double recombination.

Accordingly, in a preferred embodiment the one or more modified donor part of the Bacillus host cell genome comprises at least 150 nucleotides; preferably at least 200 nucleotides; more preferably at least 250; 300; 350; 400; 450; 500; 550; 600; 650; 700; 750; 800; 850; 900; 950 or at least 1,000 nucleotides.

It is advantageous in the methods of the present invention to employ a Bacillus host cell that is unable to quickly repair the nicked target sequence(s) without integration of the modified donor part of the genome.

Accordingly, it is preferred that the Bacillus host cell provided in step A of the method of the invention comprises an inactivated non-homologous end joining (NHEJ) system; preferably the cell comprises an inactivated DNA Ligase D (LigD) and/or DNA-end-binding protein Ku; even more preferably the cell comprises inactivated ykoV (ligD) and/or ykoU (ku) genes.

### Multiplexing

In a preferred embodiment at least one genome target sequence in the host cell selected in step B has been modified by at least one insertion, deletion and/or substitution of one or more nucleotide, codon, coding sequence or regulatory sequence.

It has been shown that several genome target sequences can be mofided simultaneously by employing a guide RNA together with Cas9. Logically, it should be possible to modify several different genome target sequences simultaneously by employing different corresponding guide RNAs or RNA complexes.

Accordingly, in a preferred embodiment, at least two genome target sequences in the host cell selected in step B have been modified by at least one insertion, deletion and/or substitution of one or more nucleotide, codon, coding sequence or regulatory sequence.

### Polynucleotides

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as a mature polypeptide coding sequence, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising certain polynucleotides operably linked to one or more control sequences that direct the expression of the coding sequence.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIlIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. Other examples of regulatory sequences are those that allow for gene amplification.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as erythromycin, lincomycin, ampicillin, chloramphenicol, kanamycin, neomycin, spectinomycin, or tetracycline resistance.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMß1 permitting replication in Bacillus.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Removal or Reduction of Activity

The present invention also relates to methods of producing a mutant of a parent cell, which comprises disrupting or deleting a polynucleotide, or a portion thereof, which results in the mutant cell producing less of the encoded polypeptide than the parent cell when cultivated under the same conditions.

The mutant cell may be constructed by reducing or eliminating expression of the polynucleotide using the methods of the invention.

Modification or inactivation of the polynucleotide may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame.

An example of a convenient way to eliminate or reduce expression of a polynucleotide is based on techniques of gene replacement, gene deletion, or gene disruption.

The polypeptide-deficient mutant cells are particularly useful as host cells for expression of native and heterologous polypeptides.

The present invention is further described by the following examples that should not be construed as limiting the scope of the invention.

### Examples

### Materials & Methods:

### Strains:

Escherichia coli

One Shot™ TOP10 chemically competent E. coli cells (Invitrogen, Carlsbad, Calif.) and Stellar™ Competent cells (Clontech laboratories, Mountain View, Calif.) were used for routine plasmid constructions and propagation.

Bacillus subtilis

B. subtilis 168.DELTA.4 was used as a host for establishing Cas9-based genome editing. B. subtilis 168.DELTA.4 is derived from the B. subtilis type strain 168 (BGSC 1A1, Bacillus Genetic Stock Center, Columbus, Ohio) and has deletions in the spollAC, aprE, nprE, and amyE genes. The deletion of these four genes was performed essentially as described for B. subtilis A164.DELTA.5, which is described in detail in U.S. Pat. No. 5,891,701.

### Media:

Bacillus strains were grown on TBAB (Tryptose Blood Agar Base, Difco Laboratories, Sparks, Md., USA) or LB agar (10 g/l Tryptone, 5 g/l yeast extract, 5 g/l NaCl, 15 g/l agar) plates or in LB liquid medium (10 g/l Tryptone, 5 g/l yeast extract, 5 g/l NaCl).

To select for erythromycin resistance, agar media were supplemented with 1 μg/ml erythromycin+25 μg/ml lincomycin and liquid media were supplemented with 5 μg/ml erythromycin.

Spizizen I medium consists of 1×Spizizen salts (6 g/l KH₂PO₄, 14 g/l K₂HPO₄, 2 g/l (NH₄)₂SO₄, 1 g/l sodium citrate, 0.2 g/l MgSO₄, pH 7.0), 0.5% glucose, 0.1% yeast extract, and 0.02% casein hydrolysate.

Spizizen II medium consists of Spizizen I medium supplemented with 0.5 mM CaCl₂), and 2.5 mM MgCl₂.

MRS medium was prepared using 55 g/l Lactobacilli MRS Broth (Becton, Dickinson and Company, Franklin Lakes, N.J.) according to manufacturer's recommendation.

Preparation and Transformation of Bacillus subtilis Competent Cells:

B. subtilis 168.DELTA.4 was spread onto LB agar plates to obtain single colonies after incubation at 37° C. overnight. After overnight incubation, one colony was used to inoculate 10 ml of LB medium. The following day, approximately 500 μl of this culture was used to inoculate 50 ml Spizizen I medium containing 5 μg/ml tryptophan. Growth was monitored using a Klett densitometer. Cells were harvested immediately as they entered stationary phase and used to inoculate Spizizen II medium containing 5 μg/ml of tryptophan. The Spizizen II culture was grown for an additional 90 minutes. Cells were harvested and either immediately used for transformation or frozen in 500 μl aliquots in 15% glycerol.

To 500 μl of competent cells, 500 μl Spizizen II medium containing 2 mM EGTA was added. Two hundred fifty microliters of cell mixture was transferred to a Falcon 2059 tube. One microgram of transforming DNA was added to each tube, followed by 250 μl of LB. Two microliters of 50 μg/ml appropriate antibiotic was included in the transformation mix. Tubes were incubated at 34° C. or 37° C. on a rotational shaker set at 250 rpm for 1 hour. Transformation reactions were plated to LB agar plates containing the appropriate antibiotic. Colonies were harvested after 24 hours at 37° C. or after 48 hours at 34° C.

Examples 1-5 below outline the construction of plasmids in this work. Examples 6-9 outline the construction of cells.

### Example 1. Construction of Plasmid pBM353

Plasmid pBM353 was designed to disrupt portions of the ykoV (ligD) and ykoU (ku) genes, simultaneously in Bacillus subtilis. Since these two genes lie in the same operon, the plasmid is designed to delete the C-terminus from ykoV (keeping amino acids 1-174; total protein is 311 amino acids), and removing the first 28 amino acids from the ku gene (612 amino acid full length protein).

Genomic DNA was isolated from B. subtilis 168.DELTA.4 according a method previously described (Pitcher, D. G., N. A. Saunders, and R. J. Owen, Rapid extraction of bacterial genomic DNA with guanidium thiocyanate. Letters in Applied Microbiology, 1989. 8(4): p. 151-156). A 555 bp fragment of the B. subtilis 168.DELTA.4 chromosome was amplified by PCR using primers 1213241 and 1213243 shown below.

Primer 1213241 (SEQ ID NO: 1):
5′-gatcggatccatgaatcgtactccttctc
Primer 1213243 (SEQ ID NO: 2):
5′-aatggatgcggagaatacagccaattttcataaacgcggag

A cleavage site for restriction enzyme BamHI (bold) was incorporated into primer 1213241.

A second 530 bp fragment of the B. subtilis 168.DELTA.4 chromosome was amplified by PCR using primers 1213241 and 1213243 shown below.

Primer 1213242 (SEQ ID NO: 3):
5′-ctccgcgtttatgaaaattggctgtattctccgcatccatt
Primer 1213244 (SEQ ID NO: 4):
5′-gatcggatccccatttgctgtttgttttc

A cleavage site for restriction enzyme BamHI (bold) was incorporated into primer 1213244.

The respective DNA fragments were amplified by PCR using Phusion Hot Start II polymerase (Thermo Scientific, Grand Island, N.Y.). The PCR amplification reaction mixture contained 1 μl of 0.1 μg/μl B. subtilis 168.DELTA.4 genomic DNA, 0.5 μl of sense primer (50 pmol/μl), 0.5 μl of anti-sense primer (50 pmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 15 seconds; one cycle at 72° C. for 5 minutes; and 4° C. hold. The PCR products were purified from a 1.0% agarose (Amresco, Solon, Ohio) gel with 1×TBE buffer using the Qiagen QIAquick Gel Extraction Kit (Qiagen, Inc., Valencia, Calif.) according to the manufacturer's instructions.

The purified PCR products were used in a subsequent PCR reaction to create a single fragment using splice overlapping PCR (SOE) using Phusion Hot Start II polymerase (Thermo Scientific, Grand Island, N.Y.) as follows: The PCR amplification reaction mixture contained 0.5 μl from the purified 555 bp PCR reaction (primers 1213241/1213243), 0.5 μl from the purified 530 bp PCR reaction (primers 1213242/1213244), 0.5 μl of sense primer 1213241 (50 pmol/μl), 0.5 μl of anti-sense primer 1213244 (50 pmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 15 seconds; one cycle at 72° C. for 5 minutes; and 4° C. hold. The PCR products were purified from a 1.0% agarose (Amresco, Solon, Ohio) gel with 1×TBE buffer using the Qiagen QIAquick Gel Extraction Kit (Qiagen, Inc., Valencia, Calif.) according to the manufacturer's instructions.

Approximately, 1 μg of the purified PCR product as well as the temperature-sensitive Bacillus/E. coli shuttle vector pShV002 (U.S. Pat. No. 5,891,701) were digested with restriction enzyme BamHI, to isolate the 1030 bp insert fragment and 7689 bp vector fragment, respectively. These fragments were isolated by 1% agarose gel electrophoresis using TBE buffer followed by purification using the Qiagen QIAquick Gel Extraction Kit (Qiagen, Inc.) according to the manufacturer's instructions. The fragments were ligated using a Rapid DNA Ligation Kit (Roche Diagnostics, Mannheim, Germany, following the manufacturer's instructions. A 2 μl aliquot of the ligation was used to transform E. coli One Shot™ cells (Invitrogen) according to the manufacturer's instructions. Plasmid DNA was prepared from E. coli transformants and digested using restriction enzyme BamHI, followed by 0.7% agarose gel electrophoresis using TBE buffer and the plasmid identified as having the correct restriction pattern was designated pBM353.

### Example 2. Construction of Plasmid pBM354

A pShV002-based temperature sensitive Bacillus/E. coli shuttle vector which does not contain the restriction site for BsaI was created using site-directed mutagenesis. The following primers were used for the PCR reaction:

Primer 1213365 (SEQ ID NO: 5):
5′-gctgaataaaagatacgaagacctctcttgtatct
Primer 1213366 (SEQ ID NO: 6):
5′-agatacaagagaggtcttcgtatcttttattcagc

The plasmid was amplified by PCR using Agilent Technologies' Quickchange II XL Site-Directed Mutagenesis kit (Agilent Technologies, Santa Clara, Calif.). The PCR amplification reaction mixture contained 1 μl of 21 ng/μl pShV002, 1 μl of sense primer (50 pmol/μl), 1 μl of anti-sense primer (50 pmol/μl), 5 μl of 10× reaction buffer, 1 μl of dNTP mix (10 mM each), 3 μl Quick Solution, 37 μl water, and 1 μl (2.5 U/μl) PfuUltra HF DNA polymerase. An Eppendorf Mastercycler thermocycler was used to amplify the fragment with the following settings: One cycle at 95° C. for 1 minute; 18 cycles each at 95° C. for 50 seconds, 60° C. for 50 seconds, and 68° C. for 3 minutes, 40 seconds. The resulting PCR product was digested with restriction enzyme DpnI for 1 hour at 37° C. A 2 μl aliquot of the ligation was used to transform E. coli One Shot™ cells according to the manufacturer's instructions. Plasmid DNA was prepared from E. coli transformants and digested using restriction enzyme BsaI, followed by 0.7% agarose gel electrophoresis using TBE buffer. The plasmid identified as having the correct restriction pattern was designated pBM354.

### Example 3. Construction of Plasmid pBM363b

A synthetic DNA fragment containing the S. pyogenes cas9 gene was obtained from GeneArt (Thermo Fischer Scientific, Grand Island, N.Y.); the DNA sequence is provided in SEQ ID NO:7 encoding SEQ ID NO:8. The fragment was cloned into temperature-sensitive Bacillus/E. coli shuttle vector, pBM354, as follows.

The following primers were used for amplification of the cas9 gene:

Primer 1213801 (SEQ ID NO: 9):
5′-gaattgggtaccgggccccccctcgagtcgacatgccggtactgccg
Primer 1213802 (SEQ ID NO: 10):
5′-cgatatcaagcttatcgataccgtcgacgtgactggcgatgctgtcgg

The respective DNA fragment was amplified by PCR using the Expand High Fidelity PLUS PCR system (Roche Diagnostics, Mannheim, Germany). The PCR amplification reaction mixture contained 1 μl 0.05 μg/μl synthetic DNA, 1 μl of sense primer (50 pmol/μl), 1 μl of anti-sense primer (50 pmol/μl), 10 μl of 5×PCR buffer with 15 mM MgCl2, 1 μl of dNTP mix (10 mM each), 36. 5 μl water, and 0.75 μl (3.5 U/μl) DNA polymerase mix. An Eppendorf Mastercycler thermocycler was used to amplify the fragment with the following settings: One cycle at 94° C. for 2 minutes; 10 cycles each at 94° C. for 15 seconds, 58° C. for 30 seconds, 72° C. for 2 minutes 40 seconds; 15 cycles each at 94° C. for 15 seconds, 58° C. for 30 seconds, 72° C. for 2 minutes 40 seconds plus 5 second elongation at each successive cycle, one cycle at 72° C. for 7 minutes; and 4° C. hold. The PCR product was purified from a 0.7% agarose (Amresco, Solon, Ohio) gel with 1×TBE buffer using the Qiagen QIAquick Gel Extraction Kit (Qiagen, Inc., Valencia, Calif.) according to the manufacturer's instructions. The 5201 bp PCR fragment containing the S. pyogenes cas9 coding sequence was cloned into plasmid pBM354, which had been previously digested with restriction enzyme SaII, using Clontech In-Fusion HD Cloning System (Clontech laboratories, Inc., Mountain View, Calif.) according to manufacturer's instructions. A 2 μl aliquot of the In-Phusion mix was used to transform E. coli Stellar™ cells according to manufacturer's instructions. Plasmid DNA was prepared from E. coli transformants and verified by sequencing of the cas9 gene using gene specific primers. The plasmid identified as having the correct sequence was designated pBM363b.

### Example 4. Construction of Plasmid pBM367b

A synthetic DNA fragment containing the scBAN promoter minus its ribosome binding site, plus the trpC guide RNA was obtained from GeneArt (Thermo Fischer Scientific, Grand Island, N.Y.); the DNA sequence is shown in SEQ ID NO:11:

5′aagctttgctgtccagactgtccgctgtgtaaaaaaaaggaataaagg
ggggttgacattattttactgatatgtataatataatttgtataagaaaa
tgtattgattctcttcaagtaggttttagagctagaaatagcaagttaaa
ataaggctagtccgttatcaacttgaaaaagtggcaccgagtcggtgctt
taagctt

The fragment was cloned into temperature-sensitive Bacillus/E. coli shuttle vector pBM354 as follows:

The following primers were used for amplification of the synthetic DNA:

Primer 1216467 (SEQ ID NO: 12):
5′-cctcgaggtcgacggtatcgataagctttgctgtccagactgtc
Primer 1216468 (SEQ ID NO: 13):
5′-gctgcaggaattcgatatcaagcttaaagcaccgactcggtgcc

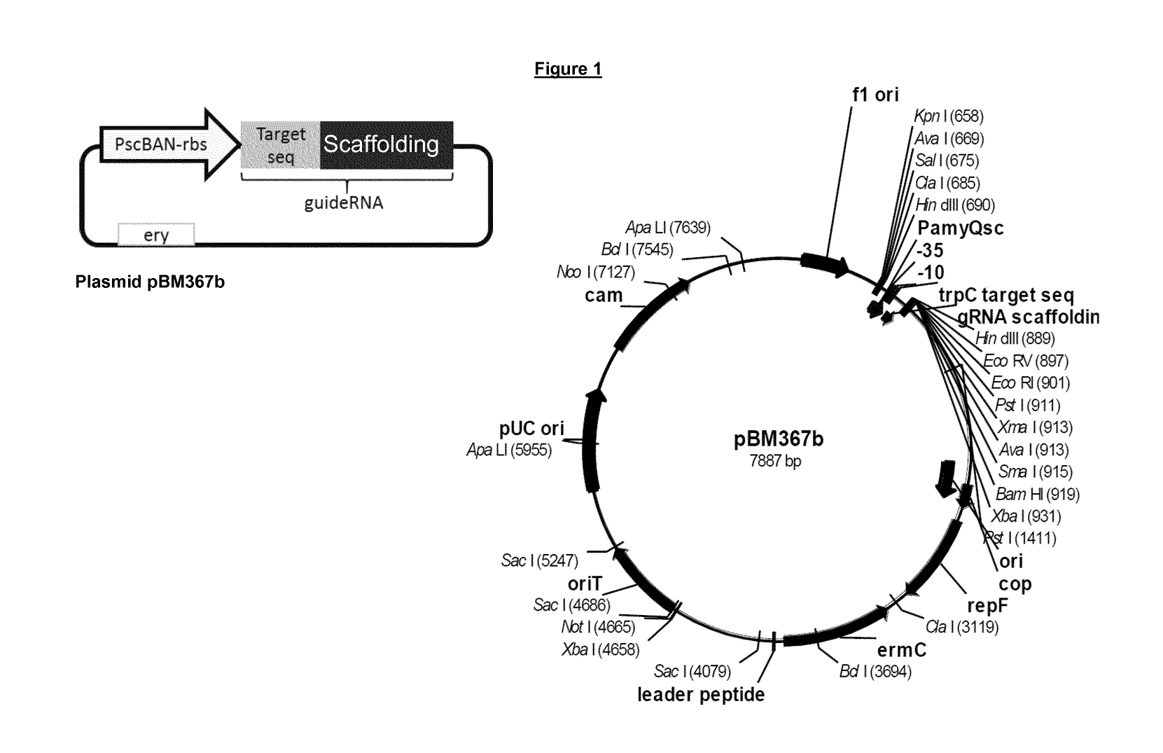

The respective DNA fragment was amplified using Illustra pure TAQ-Ready-To-Go PCR beads (GE Healthcare Biosciences, Pittsburgh, Pa.). For PCR amplification reaction, 1 μl 50 ng/μl synthetic DNA, 1 μl of sense primer (50 pmol/μl), 1 μl of anti-sense primer (50 pmol/μl), and 22 μl water was added to a PCR tube containing a Ready-To-Go PCR bead. An Eppendorf Mastercycler thermocycler was used to amplify the fragment with the following settings: One cycle at 94° C. for 2 minutes; 25 cycles each at 94° C. for 15 seconds, 58° C. for 30 seconds, 72° C. for 2 minutes, one cycle at 72° C. for 7 minutes; and 4° C. hold. The PCR product was purified from a 1.8% agarose (Amresco, Solon, Ohio) gel with 1×TBE buffer using the Qiagen QIAquick Gel Extraction Kit (Qiagen, Inc., Valencia, Calif.) according to manufacturer's instructions. The 247 bp PCR fragment comprises the scBAN promoter, minus the ribosome binding site, plus the trpC guide RNA was cloned into plasmid pBM354, which had been previously digested with restriction enzyme HindIII, using Clontech In-Fusion HD Cloning System (Clontech laboratories, Inc., Mountain View, Calif.) according to manufacturer's instructions. A 2 μl aliquot of the In-fusion mix was used to transform E. coli Stellar™ cells according to the manufacturer's instructions. Plasmid DNA was prepared from E. coli transformants. DNA sequencing of one such transformant was identified as having correct DNA sequence and designated pBM367b. FIG. 1 shows a representation of the temperature-sensitive plasmid pBM367b which contains the trpC gRNA under transcriptional control of the strong PscBAN-rbs promoter and the erythromycin resistance gene (“ery” or ermC) for selection in Bacillus.

### Example 5. Construction of Plasmid, pBM373

A synthetic DNA fragment containing the B. subtilis A164 (see U.S. Pat. No. 5,891,701) trpC gene sequence with a “G” to “A” nucleotide substitution mutation in position 351 was obtained from GeneArt (Thermo Fischer Scientific, Grand Island, N.Y.). The sequence of the synthetic DNA is shown in SEQ ID NO:14. The fragment was amplified using the following PCR primers:

Primer 064659 (SEQ ID NO: 15):
5′-aaagaagaagtgaaaacactgg
Primer 064660 (SEQ ID NO: 16):
5′-gattccgctttcgctgacaagc

The 606 bp DNA fragment was amplified using Illustra pure TAQ-Ready-To-Go PCR beads (GE Healthcare Biosciences, Pittsburgh, Pa.). For PCR amplification reaction, 1 μl 50 ng/μl synthetic DNA, 1 μl of sense primer (50 pmol/μl), 1 μl of anti-sense primer (50 pmol/μl), and 22 μl water was added to a PCR tube containing a Ready-To-Go PCR bead. An Eppendorf Mastercycler thermocycler was used to amplify the fragment with the following settings: One cycle at 94° C. for 2 minutes; 25 cycles each at 94° C. for 15 seconds, 58° C. for 30 seconds, 72° C. for 40 seconds, one cycle at 72° C. for 7 minutes; and 4° C. hold. The PCR product was purified from a 1.8% agarose (Amresco, Solon, Ohio) gel with 1×TBE buffer using the Qiagen QIAquick Gel Extraction Kit (Qiagen, Inc., Valencia, Calif.) according to the manufacturer's instructions. The purified PCR fragment was cloned into pCR2.1 using the TA-TOPO Cloning Kit (Stratagene, Inc., La Jolla, Calif.) and used to transform E. coli OneShot™ competent cells according to the manufacturers' instructions (Stratagene, Inc., La Jolla, Calif.). Transformants were selected at 37° C. after 16 hours of growth on 2× yeast-tryptone (YT) agar plates supplemented with 100 μg/ml of ampicillin. Plasmid DNA from these transformants was purified using a QIAGEN robot (QIAGEN, Valencia, Calif.) according to the manufacturer's instructions and the DNA sequence of the inserts confirmed by DNA sequencing using M13 (−20) forward and M13 reverse primers (Invitrogen, Inc., Carlsbad, Calif.) The plasmid harboring the 606 bp PCR fragment was designated as plasmid pBM371.

Plasmid, pBM371 was used as the template for PCR amplification using primer pair 1216696/1216697. These primers were designed to incorporate restriction enzyme, XhoI (bold) for ease of further cloning.

Primer 1216696 (SEQ ID NO: 17):
5′-ctcgagcaaaagaaagaagaagtgaaaacactgg
Primer 1216697 (SEQ ID NO: 18):
5′-ctcgagttcgctgacaagcaaggatt

The respective DNA fragment was amplified by PCR using Phusion Hot Start II polymerase (Thermo Scientific, Grand Island, N.Y.). The PCR amplification reaction mixture contained 1 μl of 64.1 ng/μl pBM371, 0.5 μl of sense primer (50 pmol/μl), 0.5 μl of anti-sense primer (50 pmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 20 seconds; one cycle at 72° C. for 5 minutes; and 4° C. hold. The 615 bp PCR product was purified from a 1.0% agarose (Amresco, Solon, Ohio) gel with 1×TBE buffer using the Qiagen QIAquick Gel Extraction Kit (Qiagen, Inc., Valencia, Calif.) according to manufacturer's instructions.

The purified PCR fragment was cloned into pCR4 using the TOPO blunt Cloning Kit (Stratagene, Inc., La Jolla, Calif.) and used to transform E. coli OneShot™ competent cells according to the manufacturers' instructions (Stratagene, Inc., La Jolla, Calif.). Transformants were selected at 37° C. after 16 hours of growth on 2× yeast-tryptone (YT) agar plates supplemented with 100 μg of ampicillin per ml. Plasmid DNA from these transformants was purified using a QIAGEN robot (QIAGEN, Valencia, Calif.) according to the manufacturer's instructions and the DNA sequence of the inserts confirmed by DNA sequencing using M13 (−20) forward and M13 reverse primers (Invitrogen, Inc., Carlsbad, Calif.) The plasmid harboring the 608 bp PCR fragment was designated as plasmid, pBM372.

Plasmid, pBM367b, described above, was used as the target vector backbone for cloning of the donor DNA fragment described above. Plasmid pBM367b was linearized with restriction enzyme XhoI, and further treated with shrimp alkaline phosphatase according to the manufacturer's instructions (Roche Diagnostics, Mannheim, Germany). In addition, plasmid pBM372 was digested with restriction enzyme XhoI. The resulting 7887 bp vector fragment and the 609 bp insert fragment were purified from a 1.0% agarose (Amresco, Solon, Ohio) gel with 1×TBE buffer using the Qiagen QIAquick Gel Extraction Kit (Qiagen, Inc., Valencia, Calif.) according to the manufacturer's instructions. The fragments were ligated using a Rapid DNA Ligation Kit following the manufacturer's instructions. A 2 μl aliquot of the ligation was used to transform E. coli One Shot™ cells according to the manufacturer's instructions. Plasmid DNA was prepared from E. coli transformants and digested using restriction enzyme XhoI, followed by 1.0% agarose gel electrophoresis using TBE buffer and the plasmid identified as having the correct restriction pattern was designated pBM373.

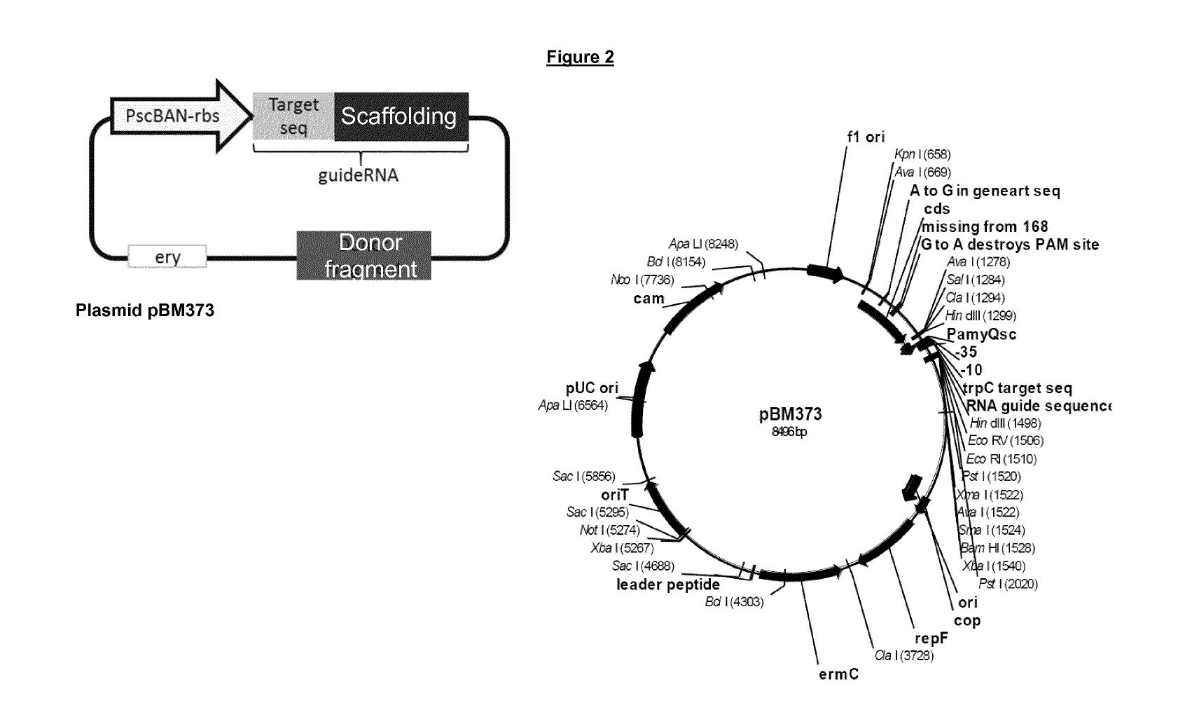

FIG. 2 shows a representation of the temperature-sensitive plasmid pBM373 which contains the trpC gRNA under transcriptional control of the strong PscBAN-rbs promoter, the erythromycin resistance gene (“ery” or ermC), and a ˜600 bp donor DNA fragment which when incorporated at the trpC locus would render the cells Trp+.

### Example 5. Construction of Plasmid pBM374

The S. pyogenes Cas9 nickase function is created by introduction of a single amino acid mutation, D10A. The following primers were designed to introduce this mutation in the S. pyogenes cas9 coding sequence found on plasmid pBM363b.

Primer 1217358 (SEQ ID NO: 19):
5′-cgacgctatttgtgccgatagctaagcctattgagtatttc
Primer 1217359 (SEQ ID NO: 20):
5′-gaaatactcaataggcttagctatcggcacaaatagcgtcg

The D10A mutation was introduced into plasmid pBM363b using Agilent Technologies' Quickchange Lightning Site-Directed Mutagenesis kit (Agilent Technologies, Santa Clara, Calif.). The PCR amplification reaction mixture contained 1 μl of 100 ng/μl pBM363b, 1 μl of sense primer (50 pmol/μl), 1 μl of anti-sense primer (50 pmol/μl), 5 μl of 10× reaction buffer, 1 μl of dNTP mix (10 mM each), 1.5 μl Quick Solution, 39.5 μl water, and 1 μl QuickChange Lightning polymerase. An Eppendorf Mastercycler thermocycler was used to amplify the fragment with the following settings: One cycle at 95° C. for 2 minutes; 18 cycles each at 95° C. for 20 seconds, 60° C. for 10 seconds, and 68° C. for 6 minutes, 20 seconds. The resulting PCR product was digested with restriction enzyme DpnI for 10 minutes at 37° C. A 2 μl aliquot of the ligation was used to transform E. coli Stellar™ cells (Clontech Laboratories, Mountain View, Calif.) according to the manufacturer's instructions. Plasmid DNA from these transformants was purified using a QIAGEN robot (QIAGEN, Valencia, Calif.) according to the manufacturer's instructions and the DNA sequence of the inserts confirmed by DNA sequencing. The plasmid harboring the cas9n coding sequence (SEQ ID NO:21) encoding the Cas9 nickase (SEQ ID NO:22) having the desired D10A mutation was designated as pBM374.

### Example 6. Construction of Strain BaC0266

To evaluate use of the type II CRISPR-Cas9 system from Streptococcus pyogenes in Bacillus, we chose to work with a Bacillus strain defective in non-homologous end joining (NHEJ). DNA damage due to double stranded breaks can be repaired in Bacillus via two pathways: error-free homologous recombination (HR) or non-homologous end joining. A double stranded break, induced by CRISPR Cas9 in a strain defective in NHEJ, would be lethal, unless repaired by homologous recombination. Two genes involved in non-homologous end joining (NHEJ) in Bacillus subtilis are annotated as ligD and ku (de Vega, M., The minimal Bacillus subtilis nonhomologous end joining repair machinery. PLoS One, 2013. 8(5): p. e64232). The ligD gene codes for a multi-functional DNA ligase D, whereas the ku gene codes for a DNA binding protein. A disruption in both genes results in a strain incapable of repairing a double stranded break by means of NHEJ. Since these two genes lie in the same operon in B. subtilis 168 both open reading frames were disrupted simultaneously.

The temperature-sensitive plasmid pBM353 was incorporated into the genome of B. subtilis 168.DELTA.4 by chromosomal integration and excision according to the method previously described (U.S. Pat. No. 5,843,720). B. subtilis 168.DELTA.4 transformants containing plasmid pBM353 were grown on TBAB supplemented with erythromycin/lincomycin at 50° C. to force integration of the vector. Desired integrants were chosen based on their ability to grow on TBAB erythromycin/lincomycin selective medium at 50° C. Integrants were then grown without selection in LB medium at 37° C. to allow excision of the integrated plasmid. Cells were plated on LB plates and screened for erythromycin-sensitivity.

Genomic DNA was prepared from several erythromycin/lincomycin sensitive isolates above accordingly to the method previously described (Pitcher, D. G., N. A. Saunders, and R. J. Owen, Rapid extraction of bacterial genomic DNA with guanidium thiocyanate. Letters in Applied Microbiology, 1989. 8(4): p. 151-156). Genomic PCR confirmed the disruption of the ykoV (ligD) and ykoU (ku) genes and the resulting strain was designated BaC0266.

### Example 7. Construction of Strain BaC0291

For proof of concept we decided to edit the trpC locus in B. subtilis 168. It has been established that the B. subtilis 168 strain is missing a 3 base pair sequence, ATT, within the trpC gene (Albertini, A. M. and A. Galizzi, The sequence of the trp operon of Bacillus subtilis 168 (trpC2) revisited. Microbiology, 1999. 145 (Pt 12): p. 3319-20). B. subtilis trpC encodes the enzyme indole-3-glycerol-phosphate synthase, which catalyzes an essential step in the biosynthesis of tryptophan. As a result of the “missing” ATT base pair sequence, B. subtilis 168 is unable to grow in minimal media unless the media are supplemented with tryptophan. Thus, we chose to edit the B. subtilis 168 genome at the trpC locus by insertion of the 3 base pairs necessary to restore the organism to trpC+.

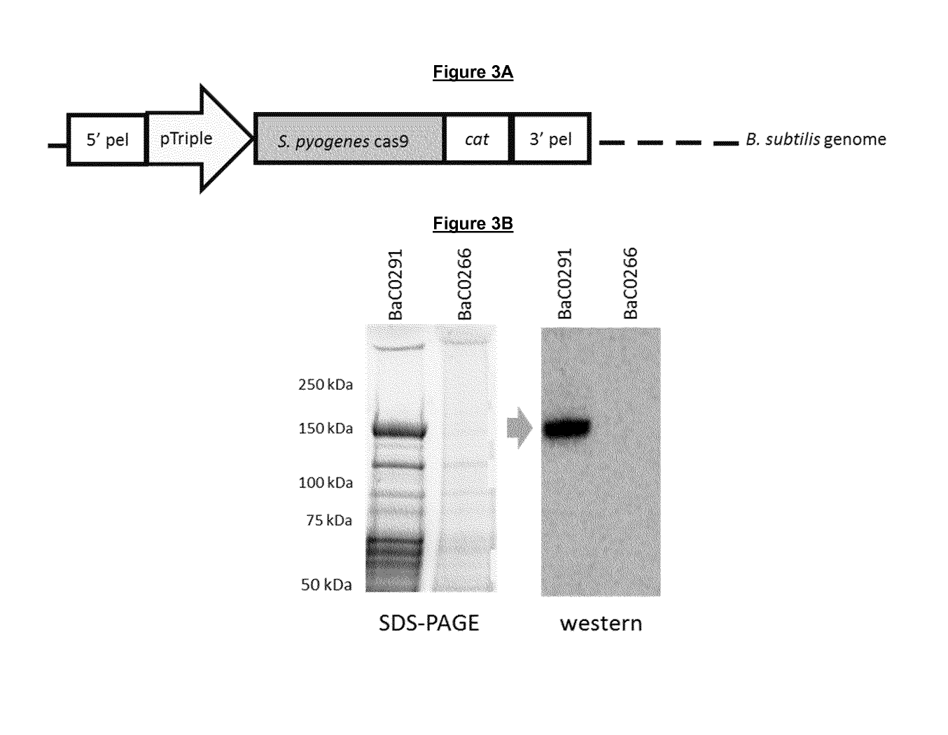

We first placed the S. pyogenes cas9 gene under transcriptional control of a strong promoter. This construct was then integrated in the B. subtilis genome in single copy at the pel locus, resulting in strain BaC0291. A schematic representation for the BaC0291 Cas9 expression construct is shown in FIG. 3A. Expression of the Cas9 protein was confirmed by analysis of cell lysates by SDS-PAGE and verified using Cas9-specific antibodies; see FIG. 3B.

Bacillus subtilis strain BaC0291 was constructed as follows: A linear integration vector-system was used for the expression cloning of the S. pyogenes cas9 gene. The linear integration construct was a PCR fusion product made by fusion of the cas9 gene between two B. subtilis homologous chromosomal regions along with a strong promoter and a chloramphenicol resistance marker. The fusion was made by SOE PCR as described in WO 2003095658. The cas9 gene was expressed under the control of a triple promoter system (as described in WO 99/43835), consisting of the promoters from B. licheniformis alpha-amylase gene (amyL), B. amyloliquefaciens alpha-amylase gene (amyQ), and the B. thuringiensis cryIIIA promoter including stabilizing sequence. The gene coding for chloramphenicol acetyl-transferase was used as selection marker (Diderichsen, B., G. B. Poulsen, and S. T. Jorgensen, A useful cloning vector for Bacillus subtilis. Plasmid, 1993. 30(3): p. 312-5). The final gene construct was integrated on the B. subtilis chromosome by homologous recombination into the pectate lyase (pel) gene locus.

The first fragment designed to amplify the 5′ pel flanking sequence with homology to the B. subtilis 168.DELTA.4 genome plus the DNA sequence for the triple promoter was amplified from B. subtilis A164 strain, MDT470, in a PCR reaction with the following primers:

Primer 1209582 (SEQ ID NO: 23):
5′-ctgcgtgtgcctacagat
Primer 1216378 (SEQ ID NO: 24):
5′-gcctattgagtatttcttatccattcggttccctcctcatttttata
gagc

The second fragment designed to contain the S. pyogenes cas9 gene was PCR amplified from plasmid pBM363b using the following primer pair:

Primer 1216377 (SEQ ID NO: 25):
5′-gctctataaaaatgaggagggaaccgaatggataagaaatactcaat
aggc
Primer 1216379 (SEQ ID NO: 26):
5′-ccgcacagcgtttttttattgattaacgcgttcagtcacctcctagc
tgactc

Finally, the third fragment designed to amplify the chloramphenicol resistance gene along with the 3′ flanking sequence with homology to the B. subtilis 168.DELTA.4 genome was amplified from MDT470 in a PCR reaction with the following primers:

Primer 1209587 (SEQ ID NO: 27):
5′-gctgaagaagctgatcgacac
Primer 1216380 (SEQ ID NO: 28):
5′-gagtcagctaggaggtgactgaacgcgttaatcaataaaaaaacgct
gtgcgg

The respective DNA fragments were amplified by PCR using Phusion Hot Start II polymerase (Thermo Scientific, Grand Island, N.Y.). The PCR amplification reaction mixture contained 1 μl of 0.1 μg/μl pBM363b plasmid DNA or 3 μl MDT470 genomic DNA, 0.5 μl of sense primer (50 pmol/μl), 0.5 μl of anti-sense primer (50 pmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 2 minutes; one cycle at 72° C. for 5 minutes; and 4° C. hold. The PCR products were purified from a 0.7% agarose (Amresco, Solon, Ohio) gel with 1×TBE buffer using the Qiagen QIAquick Gel Extraction Kit (Qiagen, Inc., Valencia, Calif.) according to manufacturer's instructions.

The purified PCR products were used in a subsequent PCR reaction to create a single fragment using splice overlapping PCR (SOE) using Phusion Hot Start II polymerase (Thermo Scientific, Grand Island, N.Y.) as follows.

The PCR amplification reaction mixture contained 1 μl 25.5 ng/μl gel purified DNA from reaction 1209582/1216378, 1 μl 32.1 ng/μl gel purified DNA from reaction 1216377/1216379, 1 μl 14.7 ng/μl gel purified DNA from reaction 1209587/1216380, 5 μl of 10× Phusion HF PCR buffer, 1 μl of dNTP mix (10 mM each), 38.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 7 minutes; one cycle at 72° C. for 5 minutes; and 4° C. hold. The PCR products were purified using the Qiagen QIAquick PCR purification Kit (Qiagen, Inc., Valencia, Calif.) according to manufacturer's instructions.

The purified PCR product (900 ng) was used to transform B. subtilis BaC0266, and transformants were selected on LB-plates containing chloramphenicol (6 μg/ml medium). Genomic DNA was prepared using the method described by Pitcher et al. (vide infra). One transformant identified by genomic PCR and further DNA sequencing of the S. pyogenes cas9 gene was chosen and named BaC0291.

### Example 8. Construction of Strain BaC0295

One microgram of BaC0291 genomic DNA was used to transform B. subtilis 168.DELTA.4 competent cells and transformants were selected on LB-plates containing chloramphenicol (6 μg/ml). One transformant identified by genomic PCR and further DNA sequencing of the cas9 gene was chosen and named BaC0295.

### Example 9. Construction of Strain BaC0298

B. subtilis strain BaC0298 was created for expression of the Cas9 D10A variant nickase-encoding gene as follows:

A linear integration vector-system was used for the expression cloning of the S. pyogenes cas9 D10A nickase gene. The linear integration construct was a PCR fusion product made by fusion of the cas9 gene between two B. subtilis homologous chromosomal regions along with a strong promoter and a chloramphenicol resistance marker. The fusion was made by SOE PCR. The gene was expressed under the control of a triple promoter system (as described in WO 99/43835), consisting of the promoters from B. licheniformis alpha-amylase gene (amyL), B. amyloliquefaciens alpha-amylase gene (amyQ), and the B. thuringiensis cryIIIA promoter including stabilizing sequence. The gene coding for Chloramphenicol acetyl-transferase was used as marker (Diderichsen, B., G. B. Poulsen, and S. T. Jorgensen, A useful cloning vector for Bacillus subtilis. Plasmid, 1993. 30(3): p. 312-5.). The final gene construct was integrated in the B. subtilis chromosome by homologous recombination into the pectate lyase gene locus.

The first fragment designed to amplify the 5′ flanking sequence with homology to the B. subtilis 168.DELTA.4 genome plus the DNA sequence for the triple promoter was amplified from B. subtilis A164 strain, MDT470, in a PCR reaction with the following primers:

1209582 (SEQ ID NO: 29):
5′-ctgcgtgtgcctacagat
1216378 (SEQ ID NO: 30):
5′-gcctattgagtatttcttatccattcggttccctcctcatttttata
gagc

The second fragment designed to contain the S. pyogenes cas9 D10A gene was PCR amplified from plasmid pBM374 using the following primer pair:

1216377 (SEQ ID NO: 31):
5′-gctctataaaaatgaggagggaaccgaatggataagaaatactcaat
aggc
1216379 (SEQ ID NO: 32):
5′-ccgcacagcgtttttttattgattaacgcgttcagtcacctcctagc
tgactc

Finally the third fragment was designed to amplify the chloramphenicol resistance gene along with the 3′ flanking sequence with homology to the B. subtilis 168.DELTA.4 genome was amplified from MDT470 in a PCR reaction with the following primers:

1209587 (SEQ ID NO: 33):
5′-gctgaagaagctgatcgacac
1216380 (SEQ ID NO: 34)
5′-gagtcagctaggaggtgactgaacgcgttaatcaataaaaaaacgct
gtgcgg

The respective DNA fragments were amplified by PCR using Phusion Hot Start II polymerase (Thermo Scientific, Grand Island, N.Y.). The PCR amplification reaction mixture contained 1 μl of 0.1 μg/μl pBM363b plasmid DNA, 0.5 μl of sense primer (50 pmol/μl), 0.5 μl of anti-sense primer (50 pmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 1 minute; one cycle at 72° C. for 5 minutes; and 4° C. hold. The PCR products was purified using the Qiagen QIAquick PCR purification Kit (Qiagen, Inc., Valencia, Calif.) according to manufacturer's instructions.

One microgram of the purified PCR product was used to transform B. subtilis BaC0266 and transformants were selected on LB-plates containing chloramphenicol (6 μg/ml). One transformant identified by genomic PCR and further DNA sequencing of the S. pyogenes cas9 gene was chosen and named BaC0298.

### Example 10. In Vitro Targeting of trpC gRNA to trpC Locus

Using an in vitro guide RNA transcription system, we validated that the trpC gRNA target sequence would target the Cas9 protein to the trpC locus and allow for DNA cleavage. To do so, we utilized the Guide-IT™ sgRNA in-vitro transcription system kit (Clontech Laboratories, Mountain View, Calif.). In this in vitro system, the guide RNA is transcribed by the T7 promoter and purified. The purified RNA molecule is then combined with recombinant Cas9 protein plus a PCR fragment containing the target DNA. The efficacy of the endonuclease complex is visualized by running the DNA fragments on an agarose gel. This kit was used to produce B. subtilis trpC sgRNA. This guide-RNA along with purified Cas9 protein (New England Laboratories, Morrisville, N.C.) was used to evaluate cleavage of target DNA amplified from the B. subtilis 168.DELTA.4 genome.

Preparation of purified sgRNA was prepared using the Guide-IT™ sgRNA in vitro transcription system as described by the manufacturer (Clontech Laboratories, Mountain View, Calif.). The following primer was used for amplification of the trpC sgRNA using the Guide-It™ sgRNA In Vitro Transcription System:

Primer 1216901 (SEQ ID NO: 35):
5′-gcggcctctaatacgactcactatagggtattgattctcttcaagta
ggttttagagctagaaatagca

The target DNA encompassing the trpC locus was PCR amplified from B. subtilis 168.DELTA.4 using the following primer pair:

1216904 (SEQ ID NO: 36):
5′-ctcgagtgtctcttctaaaagcggaa
1216905 (SEQ ID NO: 37):
5′-ctcgaggtttttttcaattccgctgg

The DNA fragment was amplified by PCR using Phusion Hot Start II polymerase (Thermo Scientific, Grand Island, N.Y.). The PCR amplification reaction mixture contained 1 μl of 0.1 μg/μl B. subtilis 168.DELTA.4 genomic DNA or 3 μl MDT470 genomic DNA, 0.5 μl of sense primer (50 pmol/μl), 0.5 μl of anti-sense primer (50 pmol/μl), 5 μl of 10× Phusion HF PCR buffer, 1 μl of dNTP mix (10 mM each), 36.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 2 minutes; one cycle at 72° C. for 5 minutes; and 4° C. hold. The PCR products were purified from a 0.7% agarose (Amresco, Solon, Ohio) gel with 1×TBE buffer using the Qiagen QIAquick Gel Extraction Kit (Qiagen, Inc., Valencia, Calif.) according to the manufacturer's instructions.

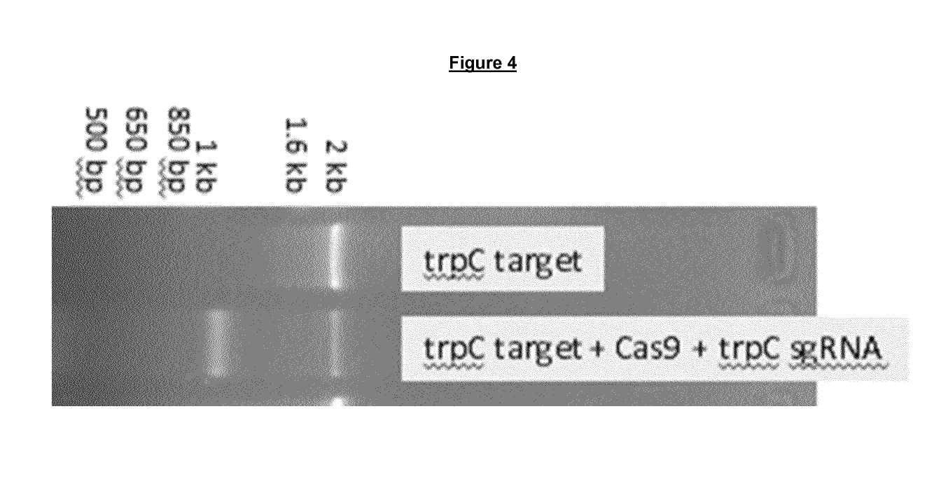

In vitro cleavage of the purified target DNA was accomplished by combining the following components in a single reaction; 6.3 μl ddH2O, 188.5 ng in-vitro transcribed trpC gRNA, 100 ng purified PCR fragment, 1 μl 10×Cas9 buffer and 1 μl 50 nm Cas9 nuclease (Clontech Laboratories, Mountain View, Calif.). The reaction was allowed to proceed after which the cleavage product was analyzed on a 0.7% agarose (Amresco, Solon, Ohio) gel with 1×TBE buffer. FIG. 4 shows the in-vitro digestion of the trpC target DNA by in-vitro transcribed trpC gRNA complexed with purified Cas9 protein. Here, the 2 kb target is cleaved into two DNA fragments as expected. The in vitro reaction does not show complete cleavage of the target DNA, likely due to the non-optimal ratio of Cas9:gRNA:DNA present in the reaction. However, the cleavage inefficiency may also be due to a non-optimal guide RNA target sequence.

### Example 11. Verification of Cas9 Expression In-Vivo

To examine expression of the Cas9 protein, BaC0291 cultures were grown overnight in MRS medium at 37° C. The following day, one ml from the overnight culture was harvested, and cells were lysed in Urea Sample buffer using lysing Matrix B (MP Biomedicals, Santa Ana, Calif.). Ten milliliters of Urea Sample buffer consists of 1 ml 10% SDS, 5.4 g urea, 250 μl 1 M Tris-HCl, 20 μl 0.5 M EDTA, pH 8.0, 500 ml beta-mercatoethanol. Cell-free lysates were subjected to SDS-PAGE using 4-15% TGX Criterion protein gels (Bio-Rad Laboratories, Hercules, Calif.). Additionally, Cas9 protein was detected using a rabbit polyclonal Cas9 antibody (Santa Cruz Biotechnology, Dallas, Tex.) with SuperSignal™ West Pico Chemiluminescent Substrate (Thermo Scientific, Grand Island, N.Y.).

### Example 12. Verification of Transcription of the Guide RNA In-Vivo

A CRISPR guide RNA was designed to target a 20 base pair sequence in the trpC locus of B. subtilis 168. The guide RNA was placed under transcriptional control of a strong promoter which had been modified by deleting the ribosome binding site to allow for RNA transcription without translation. The guide RNA expression construct was placed on a temperature-sensitive Bacillus/E. coli shuttle vector which harbors an erythromycin marker for antibiotic selection in Bacillus. The resulting plasmid was named pBM367b (see details on the construction above). The plasmid was transformed into B. subtilis BaC0266 and cultures which had been grown to exponential phase were sampled. RT-PCR was used to validate transcription of the guideRNA. To do so, total RNA was isolated using the FastRNA Pro Blue kit (MP Biomedicals, Santa Ana, Calif.) from B. subtilis cultures grown in MRS medium to a cell density reading of 190 when measured using a Klett densitometer. The total RNA was reverse transcribed using Superscript III one-step RT-PCR system with Platinum Taq (Thermo Scientific, Grand Island, N.Y.). The cDNA product was used as a template to evaluate gRNA expression by reverse transcription (RT-PCR). Two genes, recA and rpsU were included for internal controls.

The following primer pair was used to amplify recA:

1217180 (SEQ ID NO: 38):
5′-gacaagccgcgtttatcgat
1217181 (SEQ ID NO: 39):
5′-aacgacaatgtcaactgccc

The following primer pair was used to amplify rpsU:

1217182 (SEQ ID NO: 40):
5′-aatttgcgttttctagcagc
1217283 (SEQ ID NO: 41):
5′-aaaaaacgaatcgcttgaag

The following primer pair was used to amplify trpC gRNA:

1216811 (SEQ ID NO: 42):
5′-tgattctcttcaagtag
1216726 (SEQ ID NO: 43):
5′-aagcaccgactcggtgccac

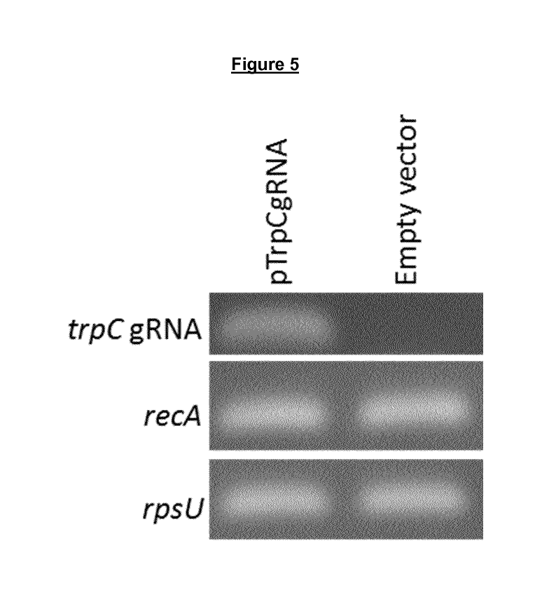

The RT-PCR reaction contained 25 μl 2× reaction mix, 1 μl 100 ng/μl template RNA, 1 μl 10 mM sense primer, 1 μl anti-sense primer, 2 μl Superscript III/Platinum Taq mix (Thermo Scientific, Grand Island, N.Y.) 20 μl ddH2O. An Eppendorf Mastercycler thermocycler was used to amplify the recA and rpsU fragments with the following settings: One cycle at 55° C. for 30 minutes; one cycle at 94° C. for 2 minutes; 40 cycles each at 94° C. for 15 seconds, 58° C. for 30 seconds, 68° C. for 12 seconds; one cycle at 68° C. for 5 minutes; and 4° C. hold. For amplification of the trpC gRNA, an Eppendorf Mastercycler thermocycler was used with the following settings: One cycle at 55° C. for 30 minutes; one cycle at 94° C. for 2 minutes; 40 cycles each at 94° C. for 15 seconds, 45° C. for 30 seconds, 68° C. for 12 seconds; one cycle at 68° C. for 5 minutes; and 4° C. hold. The resulting products were visualized on a 1.8% agarose (Amresco, Solon, Ohio) gel with 1×TBE buffer; see FIG. 5.

### Example 13. Genome Editing B. subtilis Using Cas9

After having verified all the components necessary to achieve Cas9-based editing in B. subtilis, this experiment was to determine the efficacy of the complete endonuclease complex in vivo. To do so, BaC0291 naturally competent cells were transformed with the trpC gRNA plasmid, pBM367b. The transformation reaction was plated on agar medium containing erythromycin after a period of outgrowth in non-selective medium. We expected no erythromycin resistant transformants, as a functional Cas9 protein complexed with the guide RNA would create a lethal double-stranded DNA break in the ligD-/ku-genetic background.

As expected, no erythromycin resistant transformants were identified, indicating the presence of an active endonuclease complex.

Next, the experiment was repeated with the inclusion of a ˜600 bp PCR-generated donor DNA fragment targeting the trpC locus. The donor fragment is designed to repair the Trp⁻ phenotype of the host strain, BaC0291, to Trp⁺. After a brief period of outgrowth, the transformation reaction was plated to erythromycin-containing agar plates. We expected to obtain erythromycin resistant colonies from this transformation reaction as those cells with a repaired trpC gene would no longer be a target for the active endonuclease complex. However, no erythromycin resistant transformants were obtained from the transformation reaction.

For completion, we placed a donor DNA fragment on plasmid pBM367b, resulting in plasmid pBM373. Within the donor DNA sequence, we introduced the “missing” 3 base pairs, ATT, which when incorporated would render the isolates Trp+.

In addition, a silent mutation was incorporated in this donor sequence to destroy the PAM recognition sequence. The presence of the editing template on the plasmid would ensure donor fragment availability for homologous recombination. However, even with the availability of the donor fragment, no erythromycin resistant colonies could be recovered.

We investigated whether the double stranded break induced by the active Cas9/gRNA complex could be repaired by those enzymes involved in non-homologous end joining. To do so, plasmid pBM367b, described above, was used to transform B. subtilis strain BaC0295 (B. subtilis 168.DELTA.4, pel::P3-cas9, cat). No erythromycin resistant transformants were obtained from this reaction. This result indicates the enzymes involved in non-homologous end joining are likely not expressed at sufficient levels under the conditions in which the cells were grown, and as a result cannot repair the double-stranded break induced by the active Cas9/gRNA complex.

All of the above results indicate that the double stranded break induced by the active ribonucleoprotein, Cas9 complexed with trpC gRNA, is lethal for B. subtilis, and recombination at the break site is not efficient under the conditions in which the cells were grown.

### Example 14. Genome Editing B. subtilis Using Cas9 Nickase (Cas9n)

The Cas9 protein, when complexed with a gRNA, induces a double stranded DNA break at the target specified by the guide sequence. This double stranded break is induced by two independent catalytically active domains in the protein, each cleaving one strand of DNA. A single amino acid mutation in one domain can result in a Cas9 protein with single-stranded nickase activity (Jinek, M., et al., A programmable dual-RNA-guided DNA endonuclease in adaptive bacterial immunity. Science, 2012. 337(6096): p. 816-21). Site directed mutagenesis, resulting in a single amino acid substitution, D10A, was used to create the Cas9 nickase (Cas9n). A B. subtilis Cas9n expression strain was constructed in a B. subtilis BaC0266 genetic background, as described above, and named BaC0298 (B. subtilis 168.DELTA.4, ligD-, ku-, pel::P3-cas9n, cat).

BaC0298 cells were transformed with plasmid pBM373 as described below. Erythromycin resistant transformants were grown and selected for in erythromycin containing medium, after which the cultures were shifted to non-selective medium and the non-permissive temperature. Growth at the non-permissive temperature ensured loss of the plasmid. Finally, genomic DNA was prepared from isolates and genomic PCR followed by gene specific sequencing confirmed the sequence modification at the trpC locus.

Naturally competent cells were prepared from B. subtilis strain BaC0298. Five hundred microliter aliquots of the competent cells were frozen at −80° C. in 15% glycerol. Prior to transformation, 500 μl of Spizizen II medium containing 2 mM EGTA was added to a frozen aliquot, after which 250 μl was moved to a Falcon tube. One microgram of plasmid pBM373, 250 μl LB and 2 μl 50 mg/ml erythromycin were added to the Falcon tube. Cells were grown on a rotational shaker set at 250 rpm 34° C. for 2 hours. After 2 hours, the transformation mixture was plated to agar plates containing 25 μg/ml of erythromycin and 1 μg/ml of lincomycin. Plates were put at 34° C. for 2 days. After two days, two colonies were individually grown to an optical density (OD_{600 nm}) of approximately 0.8 in LB medium containing 5 μg/ml of erythromycin, after which the cells were serially diluted and plated on agar medium containing 25 μg/ml erythromycin and 1 μg/ml lincomycin. After overnight incubation at 34° C., individual colonies were picked into 96-well microplates wherein each well contained 500 μl LB medium and incubated at 45° C. overnight. The following day, a 96-well microplate replicator was used to stamp colonies to an LB agar plate. The plate was grown at 37° C., overnight. The following day, 3 ml of LB medium was inoculated with a loop of cells from the patched colony and grown overnight at 37° C. The following day, genomic DNA was prepared using the method described by Pitcher et al. (vide infra). PCR was used to amplify the region of the genome encompassing the trpC locus using the following primer pair:

Primer 1216904 (SEQ ID NO: 44):
5′-ctcgagtgtctcttctaaaagcggaa
Primer 1218021 (SEQ ID NO: 45):
5′-ttatcttgatggtgaagcgc

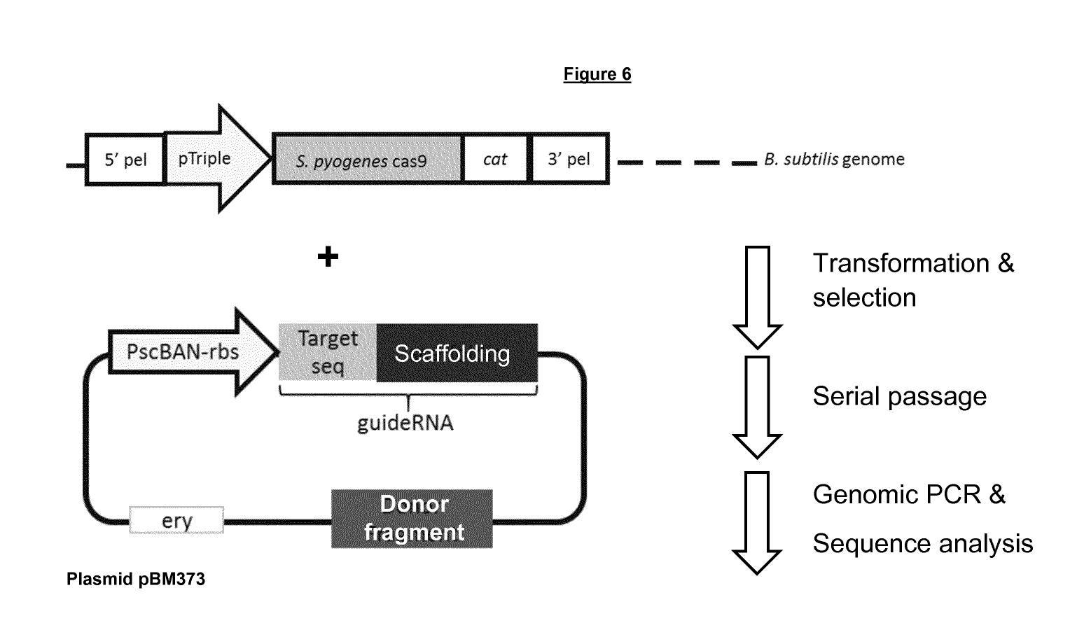

The 1771 bp DNA fragment was amplified using Phusion Hot Start II polymerase (Thermo Scientific, Grand Island, N.Y.). The PCR amplification reaction mixture contained 3 μl genomic DNA, 0.5 μl of sense primer (50 pmol/μl), 0.5 μl of anti-sense primer (50 pmol/μl), 5 μl of 10× Phusion HF PCR buffer, 1 μl of dNTP mix (10 mM each), 34.5 μl water, and 0.5 μl (2.0 U/μl) DNA polymerase mix. An Eppendorf Mastercycler thermocycler was used to amplify the fragment with the following settings: One cycle at 98° C. for 30 seconds; 25 cycles each at 98° C. for 10 seconds, 58° C. for 20 seconds, 72° C. for 40 seconds; one cycle at 72° C. for 5 minutes; and 4° C. hold. The PCR products were purified using the Qiagen QIAquick PCR Extraction Kit (Qiagen, Inc., Valencia, Calif.) according to the manufacturer's instructions. Genome editing was confirmed by sequencing analysis using trpC gene specific primers described above. A simplified schematic for the selection of isolates in shown in FIG. 6.

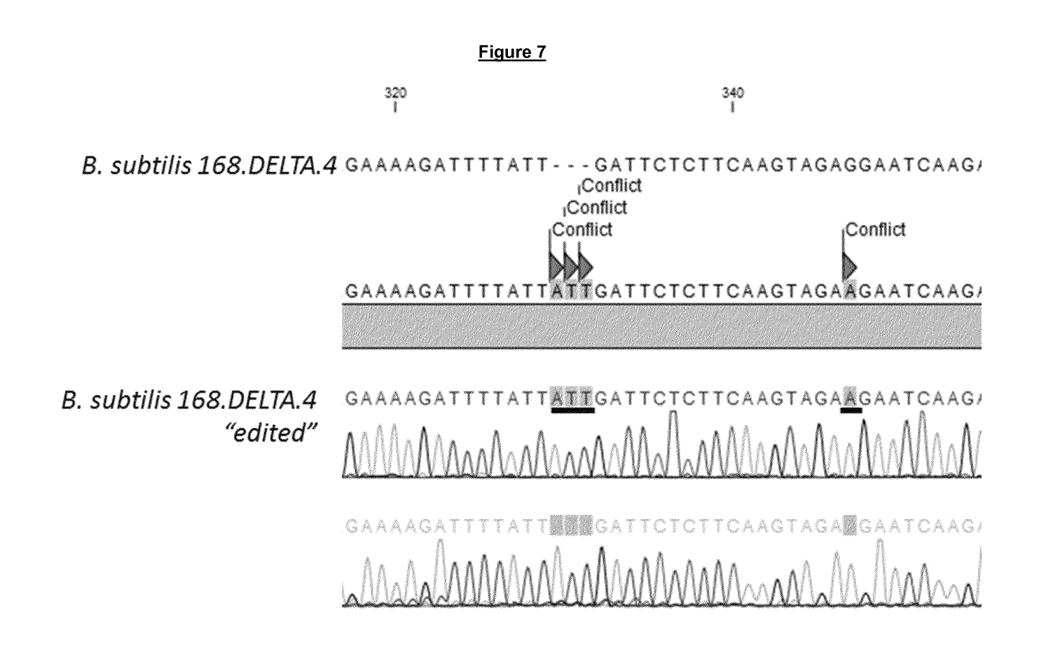

Out of 47 isolates which were evaluated, 22 had incurred the desired integration of the 3 base pair ATT sequence. A snapshot from the sequence analysis indicating the expected genetic changes for one isolate is illustrated in FIG. 7. Based on these results, one can expect effective editing of the B. subtilis genome using Cas9n, when expressed with a guide RNA, in the presence of a donor DNA fragment to a frequency of nearly 50%.

1-11. (canceled)

12. A method for modifying the genome of a Bacillus host cell, said method comprising the steps of:
A. providing a Bacillus host cell comprising:
a) at least one genome target sequence to be modified, wherein each target sequence is flanked by a functional PAM sequence for a Class-II Cas9 enzyme;
b) a variant of the Class-II Cas9 enzyme having only one active nuclease domain,
c) a single-guide RNA or a guide RNA complex for each target sequence to be modified, said RNA or RNA complex comprising:
i) a first RNA comprising 20 or more nucleotides that are at least 80% complementary to and capable of hybridizing to the at least one genome target sequence to be modified and comprising a tracr mate sequence, and
ii) a second RNA comprising a tracr sequence complementary to and capable of hybridizing with the tracr mate sequence; and
d) at least one polynucleotide construct comprising one or more modified donor part of the Bacillus host cell genome, said donor part comprising the at least one genome target sequence having the desired nucleotide modification(s) as well as at least 70 unmodified nucleotides flanking the modification(s) on each side;
wherein the 20 or more nucleotides of the first RNA hybridize with the at least one genome target sequence and wherein the variant Class-II Cas9 enzyme interacts with the single-guide RNA or the guide RNA complex and nicks the at least one genome target sequence,
whereafter the one or more modified donor part of the Bacillus host cell genome is inserted into the genome by a homologous recombination event on each side of the nick, thereby introducing the desired modification(s) into the genome; and
B. selecting a Bacillus host cell, wherein the at least one genome target sequence has been modified.

13. The method of claim 12, wherein the Bacillus host cell is selected from the group of Bacillus species consisting of Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis.

14. The method of claim 12, wherein the at least one genome target sequence to be modified comprises at least 20 nucleotides.

15. The method of claim 12, wherein the at least one genome target sequence to be modified is comprised in an open reading frame encoding a polypeptide.

16. The method of claim 12, wherein the variant of the Class-II Cas9 enzyme having only one active nuclease domain comprises a substitution of aspartic acid for alanine in the amino acid position corresponding to position 10, D10A, in the Streptomyces pyogenes Cas9 amino acid sequence shown in SEQ ID NO: 8.

17. The method of claim 12, wherein the variant of the Class-II Cas9 enzyme having only one active nuclease domain has the amino acid sequence shown in SEQ ID NO: 22.

18. The method of claim 12, wherein the single-guide RNA or RNA complex comprises a first RNA comprising 20 or more nucleotides that are at least 85% complementary to and capable of hybridizing to the at least one genome target sequence;

19. The method of claim 12, wherein the single-guide RNA or RNA complex comprises a first RNA comprising 20 or more nucleotides that are at least 90% complementary to and capable of hybridizing to the at least one genome target sequence;

20. The method of claim 12, wherein the single-guide RNA or RNA complex comprises a first RNA comprising 20 or more nucleotides that are at least 95% complementary to and capable of hybridizing to the at least one genome target sequence.

21. The method of claim 12, wherein the Bacillus host cell comprises a single-guide RNA comprising the first and second RNAs in the form of a single polynucleotide and wherein the tracr mate sequence and the tracr sequence form a stem-loop structure when hybridized with each other.

22. The method of claim 12, wherein the one or more modified donor part of the Bacillus host cell genome comprises at least 150 nucleotides.

23. The method of claim 12, wherein the one or more modified donor part of the Bacillus host cell genome comprises at least 350 nucleotides.

24. The method of claim 12, wherein the one or more modified donor part of the Bacillus host cell genome comprises at least 750 nucleotides.

25. The method of claim 12, wherein the one or more modified donor part of the Bacillus host cell genome comprises at least 1000 nucleotides.

26. The method of claim 12, wherein at least one genome target sequence in the host cell selected in step B has been modified by at least one insertion, deletion and/or substitution of one or more nucleotide, codon, coding sequence or regulatory sequence.

27. The method of claim 12, wherein at least two genome target sequences in the host cell selected in step B have been modified by at least one insertion, deletion and/or substitution of one or more nucleotide, codon, coding sequence or regulatory sequence.

28. The method of claim 12, wherein the Bacillus host cell provided in step A comprises an inactivated non-homologous end joining (NHEJ) system; preferably the cell comprises an inactivated DNA Ligase D (LigD) and/or DNA-end-binding protein Ku; even more preferably the cell comprises inactivated ykoV (ligD) and/or ykoU (ku) genes.


SEQUENCE LISTING (45 sequences)

SEQ ID NO: 1 - DNA, 29 bp, Artificial sequence
  Primer 1213241
gatcggatccatgaatcgtactccttctc

SEQ ID NO: 2 - DNA, 41 bp, Artificial sequence
  Primer 1213243
aatggatgcggagaatacagccaattttcataaacgcggag

SEQ ID NO: 3 - DNA, 41 bp, Artificial sequence
  Primer 1213242
ctccgcgtttatgaaaattggctgtattctccgcatccatt

SEQ ID NO: 4 - DNA, 29 bp, Artificial sequence
  Primer 1213244
gatcggatccccatttgctgtttgttttc

SEQ ID NO: 5 - DNA, 35 bp, Artificial sequence
  Primer 1213365
gctgaataaaagatacgaagacctctcttgtatct

SEQ ID NO: 6 - DNA, 35 bp, Artificial sequence
  Primer 1213366
agatacaagagaggtcttcgtatcttttattcagc

SEQ ID NO: 7 - DNA, 5153 bp, Streptomyces pyogenes
  CDS (579)..(4682): Encodes the Cas9 enzyme.
gtcgacatgccggtactgccgggcctcttgcgggattacgaaatcatcctgtggagcttagtaggtttagcaagatggcagcgcctaaatgtagaatgataaaaggattaagagattaatttccctaaaaatgataaaacaagcgttttgaaagcgcttgtttttttggtttgcagtcagagtagaatagaagtatcaaaaaaagcaccgactcggtgccactttttcaagttgataacggactagccttattttaacttgctatgctgttttgaatggttccaacaagattattttataacttttataacaaataatcaaggagaaattcaaagaaatttatcagccataaaacaatacttaatactatagaatgataacaaaataaactactttttaaaagaattttgtgttataatctatttattattaagtattgggtaatattttttgaagagatattttgaaaaagaaaaattaaagcatattaaactaatttcggaggtcattaaaactattattgaaatcatcaaactcattatggatttaatttaaactttttattttaggaggcaaaaatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaagtatattttagatgaagattatttcttaataactaaaaatatggtataatactcttaataaatgcagtaatacaggggcttttcaagactgaagtctagctgagacaaatagtgcgattacgaaattttttagacaaaaatagtctacgaggttttagagctatgctgttttgaatggtcccaaaactgagaccagtctcggaagctcaaaggtctcgttttagagctatgctgttttgaatggtcccaaaacttcagcacactgagacttgttgagttccatgttttagagctatgctgttttgaatggactccattcaacattgccgatgataacttgagaaagagggttaataccagcagtcggataccttcctattctttctgttaaagcgttttcatgttataataggcaaaagaagagtagtgtgatcgtccattccgacagcatcgccagtcacgtcgac

SEQ ID NO: 8 - protein, 1368 aa, Streptomyces pyogenes [sequence omitted]

SEQ ID NO: 9 - DNA, 47 bp, Artificial sequence
  Primer 1213801
gaattgggtaccgggccccccctcgagtcgacatgccggtactgccg

SEQ ID NO: 10 - DNA, 48 bp, Artificial sequence
  Primer 1213802
cgatatcaagcttatcgataccgtcgacgtgactggcgatgctgtcgg

SEQ ID NO: 11 - DNA, 205 bp, Artificial sequence
  A synthetic DNA fragment containing the scBAN promoter minus its ribosome binding site, plus the trpC guide RNA.
aagctttgctgtccagactgtccgctgtgtaaaaaaaaggaataaaggggggttgacattattttactgatatgtataatataatttgtataagaaaatgtattgattctcttcaagtaggttttagagctagaaatagcaagttaaaataaggctagtccgttatcaacttgaaaaagtggcaccgagtcggtgctttaagctt

SEQ ID NO: 12 - DNA, 44 bp, Artificial sequence
  Primer 1216467
cctcgaggtcgacggtatcgataagctttgctgtccagactgtc

SEQ ID NO: 13 - DNA, 44 bp, Artificial sequence
  Primer 1216468
gctgcaggaattcgatatcaagcttaaagcaccgactcggtgcc

SEQ ID NO: 14 - DNA, 753 bp, Artificial sequence
  A synthetic DNA fragment containing the B. subtilis A164 (see U.S. Pat. No. 5,891,701) trpC gene sequence with a “G” to “A” nucleotide substitution mutation in position 351.
atgcttgaaaaaatcatcaaacaaaagaaagaagaagtgaaaacactggttctgccggtagagcagcctttcgagaaacgttcatttaaggaggcgctggcaagcccgaatcggtttatcgggttgattgccgaagtgaagaaagcatcgccgtcaaaagggcttattaaagaggattttgtacctgtgcagattgcaaaagactatgaggctgcgaaggcagatgcgatttccgttttaacagacaccccgttttttcaaggggaaaacagctatttatcagacgtaaagcgtgctgtttcgattcctgtacttagaaaagattttattattgattctcttcaagtagaagaatcaagaagaatcggagcggatgccatattgttaatcggcgaggtgcttgatcccttacaccttcatgaattatatcttgaagcaggtgaaaaggggatggacgtgttagtggaggttcatgatgcatcaacgctagaacaaatattgaaagtgttcacacccgacattctcggcgtaaataatcgaaacctaaaaacgtttgaaacatctgtaaagcagacagaacaaatcgcatctctcgttccgaaagaatccttgcttgtcagcgaaagcggaatcggttctttagaacatttaacatttgtcaatgaacatggggcgcgagctgtacttatcggtgaatcattgatgagacaaacttctcagcgtaaagcaatccatgctttgtttagggagtga

SEQ ID NO: 15 - DNA, 22 bp, Artificial sequence
  Primer 064659
aaagaagaagtgaaaacactgg

SEQ ID NO: 16 - DNA, 22 bp, Artificial sequence
  Primer 064660
gattccgctttcgctgacaagc

SEQ ID NO: 17 - DNA, 34 bp, Artificial sequence
  Primer 1216696
ctcgagcaaaagaaagaagaagtgaaaacactgg

SEQ ID NO: 18 - DNA, 26 bp, Artificial sequence
  Primer 1216697
ctcgagttcgctgacaagcaaggatt

SEQ ID NO: 19 - DNA, 41 bp, Artificial sequence
  Primer 1217358
cgacgctatttgtgccgatagctaagcctattgagtatttc

SEQ ID NO: 20 - DNA, 41 bp, Artificial sequence
  Primer 1217359
gaaatactcaataggcttagctatcggcacaaatagcgtcg

SEQ ID NO: 21 - DNA, 5153 bp, Artificial sequence
  The synthetic Cas9 nickase (cas9n) coding sequence.
  CDS (579)..(4682): Synthetic sequence encoding the Cas9 nickase (cas9n).
gtcgacatgccggtactgccgggcctcttgcgggattacgaaatcatcctgtggagcttagtaggtttagcaagatggcagcgcctaaatgtagaatgataaaaggattaagagattaatttccctaaaaatgataaaacaagcgttttgaaagcgcttgtttttttggtttgcagtcagagtagaatagaagtatcaaaaaaagcaccgactcggtgccactttttcaagttgataacggactagccttattttaacttgctatgctgttttgaatggttccaacaagattattttataacttttataacaaataatcaaggagaaattcaaagaaatttatcagccataaaacaatacttaatactatagaatgataacaaaataaactactttttaaaagaattttgtgttataatctatttattattaagtattgggtaatattttttgaagagatattttgaaaaagaaaaattaaagcatattaaactaatttcggaggtcattaaaactattattgaaatcatcaaactcattatggatttaatttaaactttttattttaggaggcaaaaatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatcacattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactgaagtatattttagatgaagattatttcttaataactaaaaatatggtataatactcttaataaatgcagtaatacaggggcttttcaagactgaagtctagctgagacaaatagtgcgattacgaaattttttagacaaaaatagtctacgaggttttagagctatgctgttttgaatggtcccaaaactgagaccagtctcggaagctcaaaggtctcgttttagagctatgctgttttgaatggtcccaaaacttcagcacactgagacttgttgagttccatgttttagagctatgctgttttgaatggactccattcaacattgccgatgataacttgagaaagagggttaataccagcagtcggataccttcctattctttctgttaaagcgttttcatgttataataggcaaaagaagagtagtgtgatcgtccattccgacagcatcgccagtcacgtcgac

SEQ ID NO: 22 - protein, 1368 aa, Artificial sequence
  Synthetic Construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - DNA, 18 bp, Artificial sequence
  Primer 1209582
ctgcgtgtgcctacagat

SEQ ID NO: 24 - DNA, 51 bp, Artificial sequence
  Primer 1216378
gcctattgagtatttcttatccattcggttccctcctcatttttatagagc

SEQ ID NO: 25 - DNA, 51 bp, Artificial sequence
  Primer 1216377
gctctataaaaatgaggagggaaccgaatggataagaaatactcaataggc

SEQ ID NO: 26 - DNA, 53 bp, Artificial sequence
  Primer 1216379
ccgcacagcgtttttttattgattaacgcgttcagtcacctcctagctgactc

SEQ ID NO: 27 - DNA, 21 bp, Artificial sequence
  Primer 1209587
gctgaagaagctgatcgacac

SEQ ID NO: 28 - DNA, 53 bp, Artificial sequence
  Primer 1216380
gagtcagctaggaggtgactgaacgcgttaatcaataaaaaaacgctgtgcgg

SEQ ID NO: 29 - DNA, 18 bp, Artificial sequence
  Primer 1209582
ctgcgtgtgcctacagat

SEQ ID NO: 30 - DNA, 51 bp, Artificial sequence; identical to SEQ ID NO: 24
  Primer 1216378

SEQ ID NO: 31 - DNA, 51 bp, Artificial sequence; identical to SEQ ID NO: 25
  Primer 1216377

SEQ ID NO: 32 - DNA, 53 bp, Artificial sequence; identical to SEQ ID NO: 26
  Primer 1216379

SEQ ID NO: 33 - DNA, 21 bp, Artificial sequence
  Primer 1209587
gctgaagaagctgatcgacac

SEQ ID NO: 34 - DNA, 53 bp, Artificial sequence; identical to SEQ ID NO: 28
  Primer 1216380

SEQ ID NO: 35 - DNA, 69 bp, Artificial sequence
  Primer 1216901
gcggcctctaatacgactcactatagggtattgattctcttcaagtaggttttagagctagaaatagca

SEQ ID NO: 36 - DNA, 26 bp, Artificial sequence
  Primer 1216904
ctcgagtgtctcttctaaaagcggaa

SEQ ID NO: 37 - DNA, 26 bp, Artificial sequence
  Primer 1216905
ctcgaggtttttttcaattccgctgg

SEQ ID NO: 38 - DNA, 20 bp, Artificial sequence
  Primer 1217180
gacaagccgcgtttatcgat

SEQ ID NO: 39 - DNA, 20 bp, Artificial sequence
  Primer 1217181
aacgacaatgtcaactgccc

SEQ ID NO: 40 - DNA, 20 bp, Artificial sequence
  Primer 1217182
aatttgcgttttctagcagc

SEQ ID NO: 41 - DNA, 20 bp, Artificial sequence
  Primer 1217283
aaaaaacgaatcgcttgaag

SEQ ID NO: 42 - DNA, 17 bp, Artificial sequence
  Primer 1216811
tgattctcttcaagtag

SEQ ID NO: 43 - DNA, 20 bp, Artificial sequence
  Primer 1216726
aagcaccgactcggtgccac

SEQ ID NO: 44 - DNA, 26 bp, Artificial sequence
  Primer 1216904
ctcgagtgtctcttctaaaagcggaa

SEQ ID NO: 45 - DNA, 20 bp, Artificial sequence
  Primer 1218021
ttatcttgatggtgaagcgc